(19) United States
(12) Patent Application Publication
(10) Pub. No.: US 2005/0074514 A1
(43) Pub. Date: Apr. 7, 2005

(54) Zero cycle molding systems, methods and apparatuses for manufacturing dosage forms

(76) Inventors: Oliver B. Anderson, Willow Grove, PA (US); William J. Stuhl, Burlington, NJ (US); William P. Buckleman, Warminster, PA (US); Harry S. Sowden, Glenside, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/677,984

(22) Filed: Oct. 2, 2003

(51) Int. Cl. A61K 9/00; B29C 47/12

(52) U.S. Cl. 425/461

(57) ABSTRACT

Systems, methods and apparatuses for manufacturing dosage forms, and to dosage forms made using such systems, methods and apparatuses are provided. A novel injection zero cycle molding module is disclosed. Such modules may be linked, preferably via a selected transfer device, into an overall system for making dosage forms.

### FIELD OF INVENTION

This invention relates generally to systems, methods and apparatuses for manufacturing dosage forms, and to dosage forms made using such systems, methods and apparatuses.

### BACKGROUND

A variety of dosage forms, such as tablets, capsules and gelcaps are known in the pharmaceutical arts. Tablets generally refer to relatively compressed powders in various shapes. One type of elongated, film-coated capsule-shaped tablet is commonly referred to as a “caplet.” Capsules are typically manufactured using a two-piece gelatin shell formed by dipping a steel rod into gelatin so that the gelatin coats the end of the rod. The gelatin is hardened into two half-shells and the rod extracted. The hardened half-shells are then filled with a powder and the two halves joined together to form the capsule. (See generally, Howard C. Ansel et al., Pharmaceutical Dosage Forms and Drug Delivery Systems (7th Ed. 1999).)

Examples of alternative processes for producing hard shell capsules from gelatin materials are taught in U.S. Pat. Nos. 4,738,817 and 4,576,284. The '817 patent describes an injection molded pharmaceutical capsule of gelatin having a cap member, a body member, means to form a plurality of compartments therein, and means for locking the cap and body members together to form a tamper-resistant connection. The '284 patent describes a hard shell capsule having a body part and a cap part that is joinable with the body part. The cap part is die-molded or extruded as a stopper directly into the open end of the body after the body has been filled, so as to seal the contents in the capsule. Neither of these patents describes processes for providing a molded coating over a compressed tablet. Additionally, the patents lack any disclosure as to the use of insulative materials in and around a nozzle for introducing the gelatin material into the mold cavities.

Gelatin-coated tablets, commonly known as geltabs and gelcaps, are an improvement on gelatin capsules and typically comprise a tablet coated with a gelatin shell. Several well known examples of gelcaps are McNeil Consumer Healthcare's acetaminophen based products sold under the trade name Tylenol®. U.S. Pat. Nos. 4,820,524; 5,538,125; 5,228,916; 5,436,026; 5,679,406; 5,415,868; 5,824,338; 5,089,270; 5,213,738; 5,464,631; 5,795,588; 5,511,361; 5,609,010; 5,200,191; 5,459,983; 5,146,730; 5,942,034 describe geltabs and gelcaps and methods and apparatuses for making them. Conventional methods for forming gelcaps are generally performed in a batchwise manner using a number of stand-alone machines operating independently. Such batch processes typically include the unit operations of granulating, drying, blending, compacting (e.g., in a tablet press), gelatin dipping or enrobing, drying, and printing.

Unfortunately, some of the processes have certain drawbacks. For example, because these systems are batch processes, each of the various apparatuses employed is typically housed in a separate clean room that must meet FDA standards. This requires a relatively large amount of capital in terms of both space and machinery. PCT publications WO03/028990 and WO03/028619 disclose a process that would increase and streamline production rates would therefore provide many economic benefits including a reduction in the size of facilities needed to mass-produce pharmaceutical products. These inventions advantageously create a continuous operation process, as opposed to a batch process, for formation of gelcaps and other dosage forms. Furthermore, gel dipping and drying operations are in general relatively time consuming. These previously disclosed inventions advantageously create a process that simplifies the gelatin coating operation in particular and reduces drying time. Additionally, current equipment for making gelcaps and geltabs is designed to produce these forms only according to precise specifications of size and shape. These previously disclosed inventions advantageously create a more versatile method and apparatus, which could be used to produce a variety of dosage forms to deliver pharmaceuticals, nutritionals, and/or confections.

Accordingly, applicants have previously discovered that a wide variety of dosage forms, including compressed tablets, gelcaps, chewable tablets, liquid fill tablets, high potency dosage forms, and the like, some of which in and of themselves are novel, can be made using unique operating modules. Each operating module performs distinct functions, and therefore may be used as a stand-alone unit to make certain dosage forms. Alternatively, two or more of the same or different operating modules may be linked together to form a continuous process for producing other dosage forms. In essence, a “mix and match” system for the production of dosage forms is provided by the present invention. Preferably, the operating modules may be linked together as desired to operate as a single continuous process. An apparatus for continuous manufacturing of pharmaceutical dosage forms was disclosed in published PCT application WO 03/028619, including a thermal cycle molding module useful for forming a shell or coating over a dosage form or portion of a dosage form, or for producing a molded dosage form per se. In a particular embodiment, the thermal cycle molding module of WO 03/028619 was used for molding a gelatin-based coating or shell onto a solid dosage form, e.g. tablet. The original method included a heating step, in which the mold was heated to about 50-75° C. prior to injecting the gelatin based shell material. Followed by a cooling step to set the shell material in the mold. The present inventors have discovered an improved molding process and apparatus that produces the desired coated dosage forms without the need for a thermal cycle molding module.

U.S. Pat. No. 6,609,902 discloses the use of an injection molding nozzle having a tip retainer that is significantly more thermally conductive than the nozzle tip. In preferred embodiments, the retainer is made of a highly conductive beryllium copper alloy, while the nozzle tip is made from a less conductive material, such as stainless steel, tool steel or carbide. The nozzle tip is described as typically having a thermal conductivity in the range from 10 to 95 W/m-K (69 BTU-in/ft²-hr-° F. to 658 BTU-in/ft²-hr-° F.).

### SUMMARY OF THE INVENTION

The present invention relates to an apparatus for making coated dosage forms by molding flowable material. In one embodiment, the apparatus has a mold plate and a retention plate that define a mold cavity, which encloses a core, such as a compressed tablet, or a hard gelatin capsule, or other substantially solid form. A flow path is defined by an interior surface of the mold plate and the core to be coated. The apparatus further includes a nozzle assembly for introducing a flowable material into the mold cavity to coat at least a first portion of the core. The nozzle assembly has a nozzle tip and valve body comprising a valve stem and valve stem tip, wherein at least a portion of the valve stem tip or nozzle tip are constructed from or coated with a thermally insulative material. In one embodiment, both the valve stem tip and nozzle tip are constructed from material having low thermal conductivity. One embodiment uses a material having a thermal conductivity at 23° C. not greater than 2 BTU-in/ft²-hr-° F. In an alternative embodiment, the valve body is constructed from a material having at least high thermal conductivity. One embodiment uses a material having thermal conductivity at 23° C. of at least 1200 BTU-in/ft²-hr-° F. In a still further embodiment, the apparatus has a plurality of mold plates and retention plates that, when joined together, form a plurality of mold cavities, said plurality of mold plates and retention plates being rotationally mounted onto said apparatus. In one embodiment, the flowable material comprises a gelatin (e.g. the flowable material may be a gelatin-based solution further comprising various additional materials, such as auxiliary film-formers, plasticizers, colorants, antimicrobials, and the like) and at least one mold plate is made from a material having good thermal conductivity and is continually maintained during molding operations at a temperature below the softening point for the selected flowable material. The core can be in the form of a compressed tablet.

In an alternative embodiment, the present invention relates to an apparatus having a first mold plate and a second mold plate. The first mold plate and second plate define a mold cavity for enclosing a core and having a flow path defined at least in part by an interior surface of said first mold plate and the core to be coated. The apparatus further includes nozzle assemblies in said first mold plate and said second mold for introducing a flowable material into said mold cavity to coat at least a portion of said core with said flowable material. Each of the nozzle assemblies has a nozzle tip and valve body comprising a valve stem and valve stem tip, wherein at least a portion of the valve stem tip or nozzle tip are constructed from or coated with a thermally insulative material. The valve stem can be constructed from a material having at least high thermal conductivity. For instance, the valve stem can be constructed from a material having thermal conductivity at 23° C. of at least 1200 BTU-in/ft²-hr-° F. Both the nozzle tip and valve stem tip can be constructed from or coated with a material having a thermal conductivity at 23° C. not greater than 2 BTU-in/ft²-hr-° F.

In an alternative embodiment, the present invention relates to the apparatus described above plus a second mold assembly having a second mold cavity for enclosing a second core and a second nozzle assembly having a second nozzle tip and a second valve stem tip for introducing a second flowable material. At least a portion of the second nozzle tip or second valve stem tip is constructed from or coated with a thermally insulative material.

The present invention further relates to a method for making a dosage form by providing a core within a mold cavity formed between a mold plate and a retention plate and injecting a flowable material through a nozzle assembly into the mold cavity to coat at least a first portion of the core with the flowable material. The nozzle assembly has a nozzle tip and valve stem tip, wherein at least a portion of the valve stem tip or nozzle tip is constructed from or coated with a thermally insulative material.

The present invention further relates to the foregoing method plus the steps of separating the mold plate and retention plate, rotating the mold plate into alignment with a second mold plate, sealing the mold plate and second mold plate to enclose the partially coated core within a second mold cavity, and injecting a flowable material through a second nozzle assembly into said mold cavity to coat at least a second portion of said core with said flowable material. The second nozzle assembly has a second nozzle tip and second valve stem tip, wherein at least a portion of the second valve stem tip or second nozzle tip is constructed from or coated with a thermally insulative material.

The present invention further relates to a method for making a dosage form by providing a core within a mold cavity formed between a first mold plate and a second mold plate and injecting a flowable material through a nozzle assembly into the mold cavity to coat at least a first portion of the core with the flowable material. The nozzle assembly has a nozzle tip and valve stem tip, wherein at least a portion of the valve stem tip or nozzle tip is constructed from or coated with a thermally insulative material.

The present invention further relates to a dosage form produced according to the above methods and having an injection-molded coating of gelatin surrounding at least a portion of the core, such as a compressed tablet. The dosage form can have a coating of hardened gelatin material with an average thickness not greater than about 400 microns. In a further embodiment, the core is a compressed tablet in the form of a caplet.

### BRIEF DESCRIPTION OF THE DRAWINGS

FIG. 1A illustrates one example of a dosage form made according to the invention.

FIG. 1B illustrates an alternative dosage form made according to the invention and the locations for measuring coating thickness of coated dosage form.

FIG. 2 is a plan view, partially schematic, of a system for manufacturing dosage forms according to the invention.

FIG. 3 is an elevational view of the system shown in FIG. 2.

FIG. 4 is top view of a portion of a compression module shown in FIG. 3.

FIG. 5 illustrates one embodiment of the zero cycle mold unit.

FIG. 6 illustrates a gripping device used in a retention assembly.

FIGS. 7A and 7B illustrate a textured mold cavity.

FIG. 8 is a cross-sectional view of an individual zero cycle molding cavity insert assembly.

FIG. 9 illustrates a molding cavity insert for the upper mold assembly.

FIG. 10 is an isometric view of an optional conductive sleeve.

FIG. 11 is a cross-sectional view of the zero cycle molding shell material flow path.

FIG. 12 illustrates one embodiment of a temperature control system for the zero cycle molding modules.

### DETAILED DESCRIPTION OF THE INVENTION

The methods, systems, and apparatuses of this invention can be used to manufacture conventional dosage forms, having a variety of shapes and sizes, as well as novel dosage forms that could not have been manufactured heretofore using conventional systems and methods. In its most general sense, the invention relates to and functions within an overall system having some or all of the following components: 1) a compression module for making compressed dosage forms from compressible powders, 2) a zero cycle molding module for applying a coating to a substrate, 3) a transfer device for transferring dosage forms from one module to another, and 4) a process for making dosage forms comprising an improved molding apparatus. Such process may be run on a continuous or indexing basis.

As used herein, the term “dosage form” applies to any solid object, semi-solid, or liquid composition, designed to contain a specific pre-determined amount (i.e. dose) of a certain ingredient, for example an active ingredient as defined below. Suitable dosage forms may be pharmaceutical drug delivery systems, including those for oral administration, buccal administration, rectal administration, topical, transdermal, or mucosal delivery, or subcutaneous implants, or other implanted drug delivery systems; or compositions for delivering minerals, vitamins and other nutraceuticals, oral care agents, flavorants, and the like. Preferably the dosage forms of the present invention are considered to be solid, however they may contain liquid or semi-solid components. In a particularly preferred embodiment, the dosage form is an orally administered system for delivering a pharmaceutical active ingredient to the gastro-intestinal tract of a human. In another preferred embodiment, the dosage form is an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient.

Suitable active ingredients for use in this invention include for example pharmaceuticals, minerals, vitamins and other nutraceuticals, oral care agents, flavorants and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products, mucolytics, muscle relaxants, osteoporosis preparations, polydimethylsiloxanes, respiratory agents, sleep-aids, urinary tract agents and mixtures thereof.

Suitable oral care agents include breath fresheners, tooth whiteners, antimicrobial agents, tooth mineralizers, tooth decay inhibitors, topical anesthetics, mucoprotectants, and the like.

Suitable flavorants include menthol, peppermint, mint flavors, fruit flavors, chocolate, vanilla, bubblegum flavors, coffee flavors, liqueur flavors and combinations and the like.

Examples of suitable gastrointestinal agents include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotadine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine.

In one embodiment of the invention, the active ingredient may be selected from bisacodyl, famotadine, ranitidine, cimetidine, prucalopride, diphenoxylate, loperamide, lactase, mesalamine, bismuth, antacids, and pharmaceutically acceptable salts, esters, isomers, and mixtures thereof.

In another embodiment, the active ingredient is selected from analgesics, anti-inflammatories, and antipyretics, e.g. non-steroidal anti-inflammatory drugs (NSAIDs), including propionic acid derivatives, e.g. ibuprofen, naproxen, ketoprofen and the like; acetic acid derivatives, e.g. indomethacin, diclofenac, sulindac, tolmetin, and the like; fenamic acid derivatives, e.g. mefanamic acid, meclofenamic acid, flufenamic acid, and the like; biphenylcarbodylic acid derivatives, e.g. diflunisal, flufenisal, and the like; and oxicams, e.g. piroxicam, sudoxicam, isoxicam, meloxicam, and the like. In a particularly preferred embodiment, the active ingredient is selected from propionic acid derivative NSAID, e.g. ibuprofen, naproxen, flurbiprofen, fenbufen, fenoprofen, indoprofen, ketoprofen, fluprofen, pirprofen, carprofen, oxaprozin, pranoprofen, suprofen, and pharmaceutically acceptable salts, derivatives, and combinations thereof. In a particular embodiment of the invention, the active ingredient may be selected from acetaminophen, acetyl salicylic acid, ibuprofen, naproxen, ketoprofen, flurbiprofen, diclofenac, cyclobenzaprine, meloxicam, rofecoxib, celecoxib, and pharmaceutically acceptable salts, esters, isomers, and mixtures thereof.

In another embodiment of the invention, the active ingredient may be selected from pseudoephedrine, phenylpropanolamine, chlorpheniramine, dextromethorphan, diphenhydramine, astemizole, terfenadine, fexofenadine, loratadine, desloratadine, cetirizine, mixtures thereof and pharmaceutically acceptable salts, esters, isomers, and mixtures thereof.

Examples of suitable polydimethylsiloxanes, which include, but are not limited to dimethicone and simethicone, are those disclosed in U.S. Pat. Nos. 4,906,478, 5,275,822, and 6,103,260, the contents of each is expressly incorporated herein by reference. As used herein, the term “simethicone” refers to the broader class of polydimethylsiloxanes, including but not limited to simethicone and dimethicone.

The active ingredient or ingredients are present in the dosage form in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. Typically, the dosage form comprises at least about 1 weight percent, preferably, the dosage form comprises at least about 5 weight percent, e.g. about 20 weight percent of a combination of one or more active ingredients. In one preferred embodiment, the core comprises a total of at least about 25 weight percent (based on the weight of the core) of one or more active ingredients.

The active ingredient or ingredients may be present in the dosage form in any form. For example, the active ingredient may be dispersed at the molecular level, e.g. melted or dissolved, within the dosage form, or may be in the form of particles, which in turn may be coated or uncoated. If the active ingredient is in form of particles, the particles (whether coated or uncoated) typically have an average particle size of about 1-2000 microns. In one preferred embodiment, such particles are crystals having an average particle size of about 1-300 microns. In another preferred embodiment, the particles are granules or pellets having an average particle size of about 50-2000 microns, preferably about 50-1000 microns, most preferably about 100-800 microns.

In embodiments where an active ingredient is contained within the core, at least a portion of the active ingredient may be optionally coated with a release-modifying coating, as known in the art. This advantageously provides an additional tool for modifying the release profile of active ingredient from the dosage form. For example, the core may contain coated particles of one or more active ingredients, in which the particle coating confers a release modifying function, as is well known in the art. Examples of suitable release modifying coatings for particles are described in U.S. Pat. Nos. 4,173,626; 4,863,742; 4,980,170; 4,984,240; 5,286,497; 5,912,013; 6,270,805; and 6,322,819. Commercially available modified release coated active particles may also be employed. Accordingly, all or a portion of one or more active ingredients in the core may be coated with a release-modifying material.

In embodiments in which it is desired for the active ingredient to be absorbed into the systemic circulation of an animal, the active ingredient or ingredients are preferably capable of dissolution upon contact with a fluid such as water, gastric fluid, intestinal fluid or the like. In one embodiment, the dissolution characteristics of at least one active ingredient meets USP specifications for immediate release tablets containing the active ingredient. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19-20 and 856 (1999). In embodiments in which at least one active ingredient is released immediately, the immediately released active ingredient is preferably contained in the shell or on the surface of the shell, e.g. in a further coating surrounding at least a portion of the shell. In another embodiment, the dissolution characteristics of one or more active ingredients are modified: e.g. controlled, sustained, extended, retarded, prolonged, delayed and the like. In a preferred embodiment in which one or more active ingredients are released in a modified manner, the modified release active or actives are preferably contained in the core.

In certain embodiments, the dosage form of the present invention comprises a core and a shell. The core of the present invention may be prepared by any suitable method, including for example compression and molding, and depending on the method by which it is made, typically comprises, in addition to the active ingredient, a variety of excipients (inactive ingredients which may be useful for conferring desired physical properties to the core or dosage form). In embodiments in which the core is prepared by compression, suitable excipients for compression include fillers, binders, disintegrants, lubricants, glidants, and the like, as well as release-modifying compressible excipients, as are well known in the art. Suitable release-modifying compressible excipients for making the core, or a portion thereof, by compression include swellable erodible hydrophilic materials, insoluble edible materials, pH-dependent polymers, and the like.

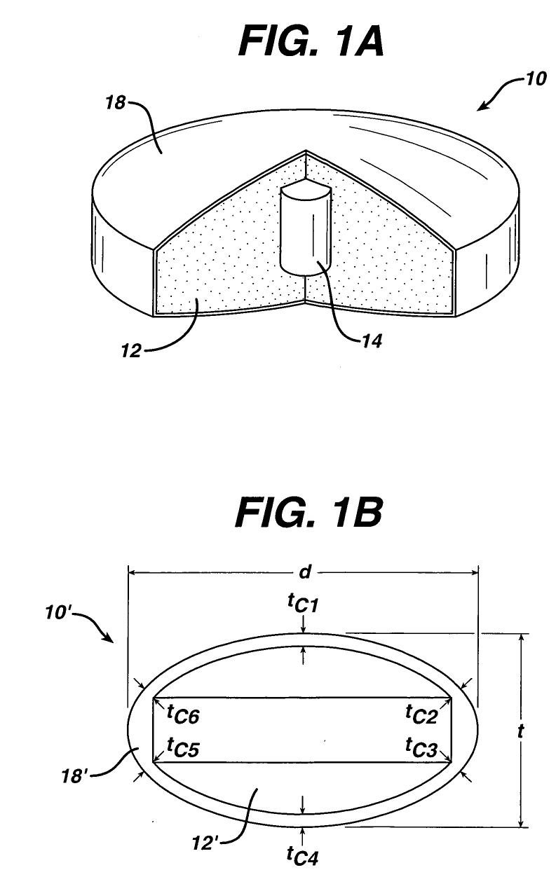

In certain embodiments, the processes described herein can produce, for example, a dosage form 10 comprising a molded coating 18 on the outside surface of a compressed core 12 also optionally containing an insert 14 as shown in FIG. 1A. FIG. 1B illustrates an alternative dosage form 10′ that may be made according to the invention comprising a molded coating 18′ over a compressed core 12′. The solid core may be of any shape, which is suitable for the oral administration of drug substances including but not limited to tablet or capsule shapes. The compressed core is, in one embodiment, a tablet such as a caplet. Suitable method of manufacturing solid cores are well known in the art such as the techniques on pages 1576-1607 of Remington's Pharmaceutical Sciences, Mack Publishing Company (Fifteenth edition), 1975 the text of which is hereby incorporated by reference. Additionally, the caplets are, in one embodiment, provided with a precoat sealant that covers the entire core before incorporation of the outer visible coating. The precoat sealant can be colored, opaque or transparent.

In certain other embodiments, the apparatus and processes described herein can produce a molded dosage form per se.

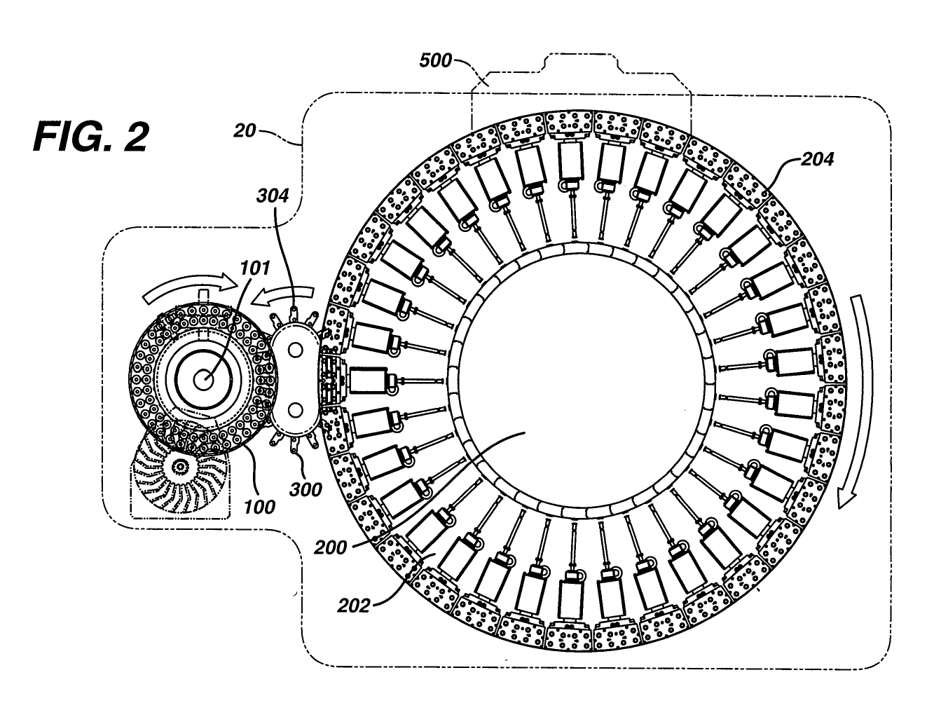

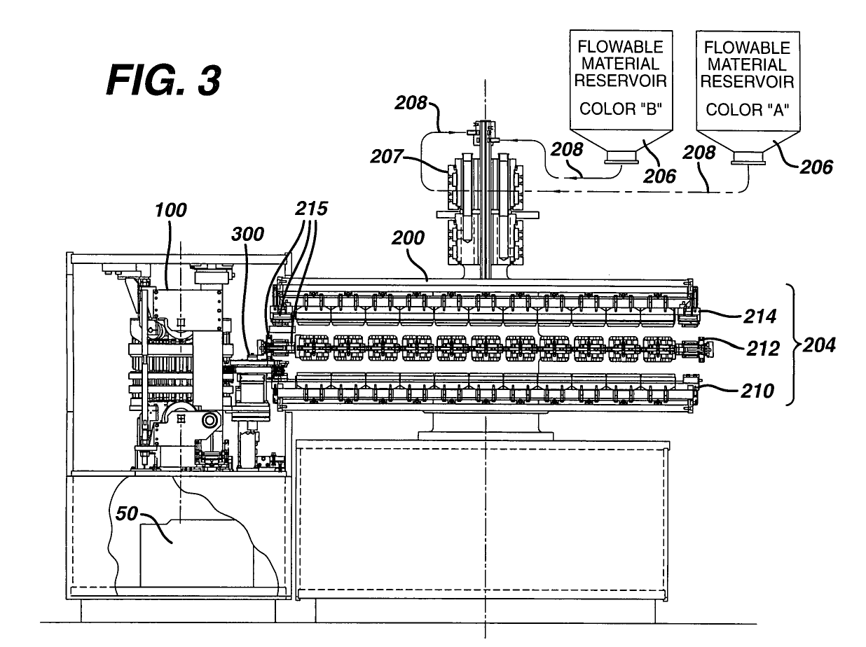

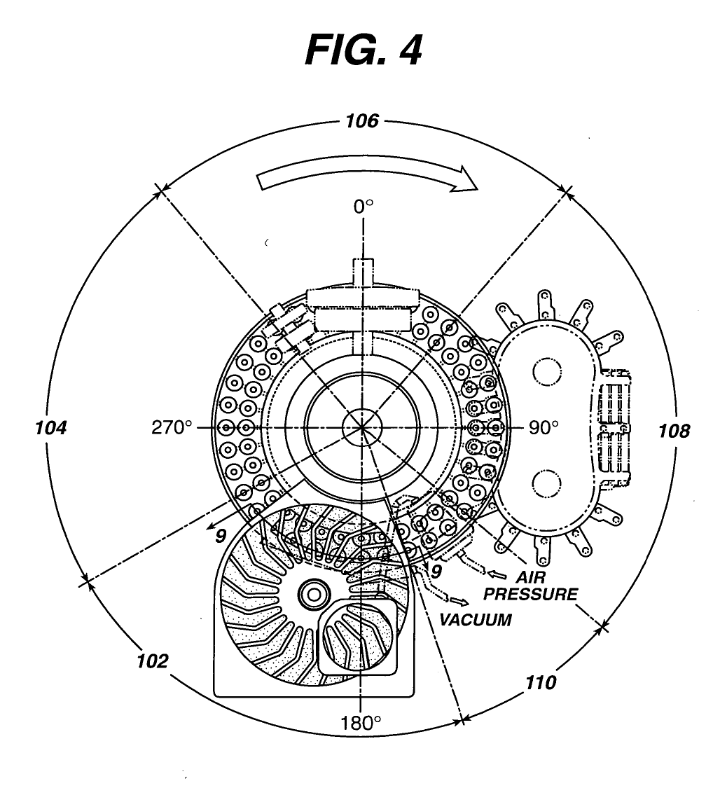

By way of overview, as illustrated in FIG. 2, system 20 comprises a compression module 100, a zero cycle molding module 200 and a transfer device 300 for transferring a compressed core made in the compression module 100 to the zero cycle molding module 200 as shown in FIGS. 3 and 4. Linkage of the compression module, transfer device, and the zero cycle molding modules in this manner results in a continuous, multi-station system. Compression is accomplished in the first module, molding of a coating around the resulting compressed core is performed in the second module, and the transfer device accomplishes transfer of the dosage form from one module to the other.

When linked in a continuous process, the operating modules can each be powered individually or jointly. In the embodiment shown in FIGS. 3 and 4, a single motor 50 powers the compression module 100, the zero cycle molding module 200, and the transfer device 300. The motor 50 can be coupled to the compression module 100, the zero cycle molding module 200 and the transfer device 300 by any conventional drive train, such as one comprising gears, gearboxes, line shafts, pulleys, and/or belts. Of course, such a motor or motors can be used to power other equipment in the process, such as the dryer (not shown) and the like.

Compression Module

The compression module 100 is generally a rotary device that performs the following functions: feeding powder to a cavity, compacting the powder into a compressed dosage form and then ejecting the compressed dosage form. Tablet presses of many kinds are known and commercially available. When the compression module is used in conjunction with the zero cycle molding module 200, upon ejection from the compression module the compressed dosage form may be transferred to the zero cycle molding module either directly or through the use of a transfer device, such as transfer device 300 described below. Optionally, an insert formed by another apparatus can be inserted into the powder in the compression module before the powder is compressed into the compressed dosage form. There are many devices available for feeding and compressing tablets. One such device is described in copending application Ser. No. 09/966,939, which is incorporated herein by reference.

In order to accomplish these functions the compression module 100 has a plurality of zones or stations, as shown schematically in FIG. 4, including a fill zone 102, a dwell zone 104, a compression zone 106, an ejection zone 108 and a purge zone 110. Thus, within a single rotation of the compression module 100 each of these functions are accomplished and further rotation of the compression module 100 repeats the cycle.

Conventional rotary tablet presses are of a single row design and contain one powder feed zone, one compression zone and one ejection zone. This is generally referred to as a single sided press since tablets are ejected from one side thereof. Presses offering a higher output version of the single row tablet press employing two powder feed zones, two tablet compression zones and two tablet ejection zones are commercially available. These presses are typically twice the diameter of the single sided version, have more punches and dies, and eject tablets from two sides thereof. They are referred to as double-sided presses.

In an embodiment of the invention, the compression module described herein is constructed with two concentric rows of punches and dies. This double row construction provides for an output equivalent to two single side presses, yet fits into a small, compact space roughly equal to the space occupied by one conventional single sided press. This also provides a simplified construction by using a single fill zone 102, a single compression zone 106, and a single ejection zone 108. Of course, a compression module with one row or more than two rows can also be constructed.

Powder is fed into die cavities in the fill zone 102. The powder can consist essentially of a medicant optionally containing various excipients, such as binders, disintegrants, lubricants, fillers and the like, as is conventional, or other particulate material of a medicinal or non-medicinal nature, such as inactive placebo blends for tableting, confectionery blends, and the like. One formulation comprises medicant, powdered wax (such as shellac wax, shellac, microcrystalline wax, polyethylene glycol, and the like), and optionally disintegrants and lubricants and is described in more detail in commonly assigned co-pending U.S. patent application Ser. No. 09/966,493, entitled “Immediate Release Tablet”, which is hereby incorporated by reference.

Suitable medicants include for example pharmaceuticals, minerals, vitamins and other nutraceuticals. Suitable pharmaceuticals include analgesics, decongestants, expectorants, antitussives, antihistamines, gastrointestinal agents, diuretics, bronchodilators, sleep-inducing agents and mixtures thereof. Preferred pharmaceuticals include acetaminophen, ibuprofen, flurbiprofen, ketoprofen, naproxen, diclofenac, aspirin, pseudoephedrine, phenylpropanolamine, chlorpheniramine maleate, dextromethorphan, diphenhydramine, famotidine, loperamide, ranitidine, cimetidine, astemizole, terfenadine, fexofenadine, loratadine, cetirizine, antacids, mixtures thereof and pharmaceutically acceptable salts thereof. In one embodiment, the medicant is selected from the group consisting of acetaminophen, ibuprofen, pseudoephedrine, dextromethorphan, diphenhydramine, chlorpheniramine, calcium carbonate, magnesium hydroxide, magnesium carbonate, magnesium oxide, aluminum hydroxide, mixtures thereof, and pharmaceutically acceptable salts thereof.

The medicant(s) is present in the dosage form in a therapeutically effective amount, which is an amount that produces the desired therapeutic response upon oral administration and can be readily determined by one skilled in the art. In determining such amounts, the particular medicant being administered, the bioavailability characteristics of the medicant, the dose regime, the age and weight of the patient, and other factors must be considered, as known in the art. In one embodiment, the compressed dosage form comprises at least about 85 weight percent of medicant, particularly a low potency medicant, such as an analgesic, including acetaminophen and ibuprofen, which may be combined with an antihistamine.

If the medicant has an objectionable taste, and the dosage form is intended to be chewed or disintegrated in the mouth prior to swallowing, the medicant may be coated with a taste masking coating, as known in the art. Examples of suitable taste masking coatings are described in U.S. Pat. No. 4,851,226, U.S. Pat. No. 5,075,114, and U.S. Pat. No. 5,489,436. Commercially available taste masked medicants may also be employed. For example, acetaminophen particles, which are encapsulated with ethylcellulose or other polymers by a coacervation process, may be used in the present invention. Coacervation-encapsulated acetaminophen may be purchased commercially from Eurand America, Inc. Vandalia, Ohio, or from Circa Inc., Dayton, Ohio.

Suitable excipients include fillers, which include water-soluble compressible carbohydrates such as dextrose, sucrose, mannitol, sorbitol, maltitol, xylitol, lactose, and mixtures thereof, water insoluble plastically deforming materials such as microcrystalline cellulose or other cellulosic derivatives, water-insoluble brittle fracture materials such as dicalcium phosphate, tricalcium phosphate, and the like; other conventional dry binders such as polyvinyl pyrrolidone, hydroxypropylmethylcellulose, and the like; sweeteners such as aspartame, acesulfame potassium, sucralose, and saccharin; lubricants, such as magnesium stearate, stearic acid, talc, and waxes; and glidants, such as colloidal silicon dioxide. The mixture may also incorporate pharmaceutically acceptable adjuvants, including, for example, preservatives, flavors, antioxidants, surfactants, and coloring agents. In one embodiment, the powder is substantially free of water-soluble polymeric binders and hydrated polymers.

Zero Cycle Molding Module

The zero cycle molding module 200 may function in one of several different ways. It may for example be used to form a shell or coating over at least part of a dosage form such as a compressed core such as a tablet. Such a coating or dosage form is made from a flowable material, alternatively primarily in liquid form. When it is in the fluid or flowable state, the flowable material may comprise a dissolved or molten component, and optionally a solvent such as for example water or organic solvents, or combinations thereof. The solvent may be partially or substantially removed by drying. In one embodiment, the zero cycle molding module is used to apply a coating of a flowable material to a compressed core made in a compression module and transferred via a transfer device also according to the invention. The coating is formed within the zero cycle molding module by injecting the flowable material into a mold assembly around the dosage form. The flowable material may or may not comprise a medicant and appropriate excipients, as desired. Alternatively, the zero cycle molding module may be used to apply a coating of flowable material to a molded dosage form, or other substrate.

Suitable flowable materials for making the core, or the shell, or a portion thereof by molding include those comprising thermoplastic materials; film formers; thickeners such as gelling polymers or hydrocolloids; low melting hydrophobic materials such as fats and waxes; non-crystallizable carbohydrates; and the like. Suitable molten components of the flowable material include thermoplastic materials, low melting hydrophobic materials, and the like. Suitable dissolved components for the flowable material include film formers, thickeners such as gelling polymers or hydrocolloids, non-crystallizable carbohydrates, and the like.

Suitable thermoplastic materials can be molded and shaped when heated, and include both water soluble and water insoluble polymers that are generally linear, not crosslinked, nor strongly hydrogen bonded to adjacent polymer chains. Examples of suitable thermoplastic materials include: thermoplastic water swellable cellulose derivatives, thermoplastic water insoluble cellulose derivatives, thermoplastic vinyl polymers, thermoplastic starches, thermoplastic polyalkylene glycols, thermoplastic polyalkylene oxides, and amorphous sugar-glass, and the like, and derivatives, copolymers, and combinations thereof. Examples of suitable thermoplastic water swellable cellulose derivatives include hydroxypropyl cellulose (HPC), hydroxypropylmethyl cellulose (HPMC), methyl cellulose (MC). Examples of suitable thermoplastic water insoluble cellulose derivatives include cellulose acetate (CA), ethyl cellulose (EC), cellulose acetate butyrate (CAB), cellulose propionate. Examples of suitable thermoplastic vinyl polymers include polyvinyl alcohol (PVA) and polyvinyl pyrrolidone (PVP). Examples of suitable thermoplastic starches are disclosed for example in U.S. Pat. No. 5,427,614. Examples of suitable thermoplastic polyalkylene glycols include polyethylene glycol. Examples of suitable thermoplastic polyalkylene oxides include polyethylene oxide having a molecular weight from about 100,000 to about 900,000 Daltons. Other suitable thermoplastic materials include sugar in the form on an amorphous glass such as that used to make hard candy forms.

Any film former known in the art is suitable for use in the flowable material of the present invention. Examples of suitable film formers include, but are not limited to, film-forming water soluble polymers, film-forming proteins, film-forming water insoluble polymers, and film-forming pH-dependent polymers. In one embodiment, the film-former for making the core or shell or portion thereof by molding may be selected from cellulose acetate, ammonia methacrylate copolymer type B, such as in U.S. Pat. No. 6,066,039, which is incorporated herein by reference, shellac, hydroxypropylmethylcellulose, and polyethylene oxide, and combinations thereof.

Suitable film-forming water soluble polymers include water soluble vinyl polymers such as polyvinyl alcohol (PVA); water soluble polycarbohydrates such as hydroxypropyl starch, hydroxyethyl starch, pullulan, methylethyl starch, carboxymethyl starch, pre-gelatinized starches, and film-forming modified starches; water swellable cellulose derivatives such as hydroxypropyl cellulose (HPC), hydroxypropylmethyl cellulose (HPMC), methyl cellulose (MC), hydroxyethylmethylcellulose (HEMC), hydroxybutylmethylcellulose (HBMC), hydroxyethylethylcellulose (HEEC), and hydroxyethyl hydroxypropylmethyl cellulose (HEMPMC); water soluble copolymers such as methacrylic acid and methacrylate ester copolymers, polyvinyl alcohol and polyethylene glycol copolymers, polyethylene oxide and polyvinylpyrrolidone copolymers; and derivatives and combinations thereof.

Suitable film-forming proteins may be natural or chemically modified, and include gelatin, whey protein, myofibrillar proteins, coagulatable proteins such as albumin, casein, caseinates and casein isolates, soy protein and soy protein isolates, zein; and polymers, derivatives and mixtures thereof.

Suitable film-forming water insoluble polymers, include for example ethylcellulose, polyvinyl alcohols, polyvinyl acetate, polycaprolactones, cellulose acetate and its derivatives, acrylates, methacrylates, acrylic acid copolymers; and the like and derivatives, copolymers, and combinations thereof.

Suitable film-forming pH-dependent polymers include enteric cellulose derivatives, such as for example hydroxypropyl methylcellulose phthalate, hydroxypropyl methylcellulose acetate succinate, cellulose acetate phthalate; natural resins, such as shellac and zein; enteric acetate derivatives such as for example polyvinyl acetate phthalate, cellulose acetate phthalate, acetaldehyde dimethylcellulose acetate; and enteric acrylate derivatives such as for example polymethacrylate-based polymers such as poly(methacrylic acid, methyl methacrylate) 1:2, which is commercially available from Rohm Pharma GmbH under the tradename, EUDRAGIT S, and poly(methacrylic acid, methyl methacrylate) 1:1, which is commercially available from Rohm Pharma GmbH under the tradename, EUDRAGIT L, and the like, and derivatives, salts, copolymers, and combinations thereof.

Any thickener known in the art is suitable for use in the flowable material of the present invention. Examples of such thickeners include but are not limited to hydrocolloids (also referred to herein as gelling polymers), clays, gelling starches, and crystallizable carbohydrates, and derivatives, copolymers and mixtures thereof.

Examples of suitable hydrocolloids (also referred to herein as gelling polymers) such as alginates, agar, guar gum, locust bean, carrageenan, tara, gum arabic, tragacanth, pectin, xanthan, gellan, maltodextrin, galactomannan, pusstulan, laminarin, scleroglucan, gum arabic, inulin, pectin, whelan, rhamsan, zooglan, methylan, chitin, cyclodextrin, chitosan. Examples of suitable clays include smectites such as bentonite, kaolin, and laponite; magnesium trisilicate, magnesium aluminum silicate, and the like, and derivatives and mixtures thereof. Examples of suitable gelling starches include acid hydrolyzed starches, and derivatives and mixtures thereof. Additional suitable thickening hydrocolloids include low-moisture polymer solutions such as mixtures of gelatin and other hydrocolloids at water contents up to about 30%, such as for example those used to make “gummi” confection forms.

Additional suitable thickeners include crystallizable carbohydrates, and the like, and derivatives and combinations thereof. Suitable crystallizable carbohydrates include the monosaccharides and the oligosaccharides. Of the monosaccharides, the aldohexoses e.g., the D and L isomers of allose, altrose, glucose, mannose, gulose, idose, galactose, talose, and the ketohexoses e.g., the D and L isomers of fructose and sorbose along with their hydrogenated analogs: e.g., glucitol (sorbitol), and mannitol are preferred. Of the oligosaccharides, the 1,2-disaccharides sucrose and trehalose, the 1,4-disaccharides maltose, lactose, and cellobiose, and the 1,6-disaccharides gentiobiose and melibiose, as well as the trisaccharide raffinose are preferred along with the isomerized form of sucrose known as isomaltulose and its hydrogenated analog isomalt. Other hydrogenated forms of reducing disaccharides (such as maltose and lactose), for example, maltitol and lactitol are also preferred. Additionally, the hydrogenated forms of the aldopentoses: e.g., D and L ribose, arabinose, xylose, and lyxose and the hydrogenated forms of the aldotetroses: e.g., D and L erythrose and threose are preferred and are exemplified by xylitol and erythritol, respectively.

In one embodiment of the invention, the flowable material comprises gelatin as a gelling polymer. Gelatin is a natural, thermogelling polymer. It is a tasteless and colorless mixture of derived proteins of the albuminous class, which is ordinarily soluble in warm water. Two types of gelatin—Type A and Type B—are commonly used. Type A gelatin is a derivative of acid-treated raw materials. Type B gelatin is a derivative of alkali-treated raw materials. The moisture content of gelatin, as well as its Bloom strength, composition and original gelatin processing conditions, determine its transition temperature between liquid and solid. Bloom is a standard measure of the strength of a gelatin gel, and is roughly correlated with molecular weight. Bloom is defined as the weight in grams required to move a half-inch diameter plastic plunger 4 mm into a 6.67% gelatin gel that has been held at 10° C. for 17 hours. In certain embodiments of the invention, the level of gelatin is from about 20% to about 50% by weight of the flowable material. In one such embodiment, the gelatin has a Bloom value from about 175 to about 325, e.g. about 250 to about 275 Bloom, or about 275 Bloom. In one embodiment the level of gelatin is from about 25% to about 45%, e.g. about 35 to about 40% by weight of the flowable material.

In certain embodiments the gelatin may comprise a mixture of skin-derived and bone-derived sources. In one particular embodiment, the flowable material is an aqueous solution comprising 17.5% 275 Bloom pork skin gelatin, 17.5% 250 Bloom Bone Gelatin, 10% polyethylene oxide, and approximately 62.9% water by weight. In another particular embodiment, the flowable material is an aqueous solution comprising 35% 275 Bloom pork skin gelatin, 1% polyethylene oxide, and approximately 62.6% water. In yet another particular embodiment, the flowable material is an aqueous solution comprising 38% 275 Bloom pork skin gelatin, and approximately 61.1% water by weight.

In certain embodiments, a coating modifier may optionally be employed in the flowable material at a level up to about 20%, e.g. up to about 12%, say from about 0.5 to about 3%, or about 10%. Suitable, coating modifiers include water-soluble or water swellable polymers such as polyalkylene oxides, hydrocolloids, and the like. Examples of polyalkylene oxides include polyethylene oxide having a molecular weight from about 200,000. Examples of hydrocolloids include xanthan gum, carrageenan, alginates, pectins, tragacanth, karaya gum, xanthan gum, carrageenan, agar and acacia.

Suitable low-melting hydrophobic materials include fats, fatty acid esters, phospholipids, and waxes. Examples of suitable fats include hydrogenated vegetable oils such as for example cocoa butter, hydrogenated palm kernel oil, hydrogenated cottonseed oil, hydrogenated sunflower oil, and hydrogenated soybean oil; and free fatty acids and their salts. Examples of suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, and stearoyl macrogol-32 glycerides. Examples of suitable phospholipids include phosphotidyl choline, phosphotidyl serene, phosphotidyl enositol, and phosphotidic acid. Examples of suitable waxes include carnauba wax, spermaceti wax, beeswax, candelilla wax, shellac wax, microcrystalline wax, and paraffin wax; fat-containing mixtures such as chocolate; and the like.

Suitable non-crystallizable carbohydrates include non-crystallizable sugars such as polydextrose, and starch hydrolysates, e.g. glucose syrup, corn syrup, and high fructose corn syrup; and non-crystallizable sugar-alcohols such as maltitol syrup.

Suitable solvents for optional use as components of the flowable material for making the core, or the shell, or a portion thereof by molding include water; polar organic solvents such as methanol, ethanol, isopropanol, acetone, and the like; and non-polar organic solvents such as methylene chloride, and the like; and mixtures thereof.

The flowable material may optionally comprise adjuvants or excipients, in which may comprise up to about 20% by weight of the flowable material. Examples of suitable adjuvants or excipients include plasticizers, detackifiers, humectants, surfactants, anti-foaming agents, colorants, flavorants, sweeteners, opacifiers, and the like. Suitable plasticizers for making the core, the shell, or a portion thereof, by molding include, but not be limited to polyethylene glycol; propylene glycol; glycerin; sorbitol; triethyl citrate; tributyl citrate; dibutyl sebecate; vegetable oils such as castor oil, rape oil, olive oil, and sesame oil; surfactants such as polysorbates, sodium lauryl sulfates, and dioctyl-sodium sulfosuccinates; mono acetate of glycerol; diacetate of glycerol; triacetate of glycerol; natural gums; triacetin; acetyltributyl citrate; diethyloxalate; diethylmalate; diethyl fumarate; diethylmalonate; dioctylphthalate; dibutylsuccinate; glyceroltributyrate; hydrogenated castor oil; fatty acids; substituted triglycerides and glycerides; and the like and/or mixtures thereof. In one embodiment, the plasticizer is triethyl citrate. In certain embodiments, the shell is substantially free of plasticizers, i.e. contains less than about 1%, say less than about 0.01% of plasticizers.

In one embodiment, the flowable material comprises less than 5% humectants, or alternately is substantially free of humectants, such as glycerin, sorbitol, maltitol, xylitol, or propylene glycol. Humectants have traditionally been included in pre-formed films employed in enrobing processes, such as that disclosed in U.S. Pat. No. 5,146,730 and U.S. Pat. No. 5,459,983, assigned to Banner Gelatin Products Corp., in order to ensure adequate flexibility or plasticity and bondability of the film during processing. Humectants function by binding water and retaining it in the film. Pre-formed films used in enrobing processes can typically comprise up to 45% water. Disadvantageously, the presence of humectant prolongs the drying process, and can adversely affect the stability of the finished dosage form. Advantageously, drying of the dosage form after it has left the zero cycle molding module not is required when the moisture content of the flowable material is less than about 5%.

The process is an improvement over the soft gelatin dipping process and differs fundamentally from hard shell gelatin capsule production. Gelatin materials, once hydrated, have a very abrupt transition temperature between the liquid phase and the solid or gel phase. This problem is particularly acute when making relatively thin coatings as typically required for healthcare dosage forms. The shell or coating produced by the inventive zero cycle molding module generally has a thickness less than 400 microns, more commonly less than 300 microns, typically less than 200 microns. Additionally, the coatings are generally at least about 25 microns thick, more commonly about 50 microns thick, 100 and 125 microns. Various combinations of thicknesses are possible depending on the dosage form requirements. A prior solution to this problem had been the use of rapid thermal cycling molds. In other words, the molds were kept warm during injection of the moldable material and then quickly cooled to promote hardening. This arrangement required extensive controls and transfer mechanisms.

Applicants discovered that the complexities of thermal cycling could be avoided if certain modifications were made to the molding modules. Through the use and positioning of selected non-conductive or thermally insulative materials, portions of the mold assemblies can be continually maintained during molding operations at a temperature below the gel or melting point of the flowable material, while portions of the mold assembly designed for the transfer and delivery of flowable material are continually maintained at relatively elevated temperatures for optimal flow properties.

Use of the zero cycle molding module advantageously avoids visible defects in the surface of the product produced. Known injection molding processes utilize sprues and runners to feed moldable material into the mold cavity. This results in product defects such as injector marks, sprue defects, gate defects, and the like. In conventional molds, sprues and runners must be broken off after solidification, leaving a defect at the edge of the part, and generating scrap. In conventional hot runner molds, sprues are eliminated, however a defect is produced at the injection point since the hot runner nozzle must momentarily contact the chilled mold cavity during injection. As the tip of the nozzle retracts it pulls a “tail” with it, which must be broken off. This defect is particularly objectionable with stringy or sticky materials. Unwanted defects of this nature would be particularly disadvantageous for swallowable dosage forms, not only from a cosmetic standpoint but functionally as well. The sharp and jagged edges would irritate or scratch the mouth, tongue and throat. Additionally, the thermal cycle molding system could produce defects as final dosage forms were ejected from the mold assemblies. The zero cycle molding module described herein avoids these problems.

Zero cycle molding module 200 generally includes a rotor 202, as shown in FIGS. 2 and 3 around which a plurality of mold units 204 are disposed. As rotor 202 revolves, mold units 204 receive compressed dosage forms, preferably from a transfer device such as transfer device 300. Each dosage form is held in position with a retaining device that grips the dosage forms around their circumference. The retaining device may be solid or segmented flexible strip provided along the upper edge of each mold cavity 288. Next, flowable material is injected into the mold units to coat the retained compressed dosage forms. After the compressed dosage forms have been coated, the coating may be further hardened or dried if required. They may be hardened within the mold units or they may be transferred to another device such as a dryer. Continued revolution of the rotor 202 repeats the cycle for each mold unit.

The zero cycle molding module 200 includes a material feed system 203 comprising at least one reservoir 206 containing the flowable material, as shown in FIG. 3, the tubing 208, the material feed plate 215. There may be a single reservoir for each mold unit, one reservoir for all the mold units, or multiple reservoirs that serve multiple mold units. In an embodiment, flowable materials of two different colors are used to make the coating, and there are two reservoirs 206, one for each color. One product of the inventive process and apparatus is a coated caplet in which two distinctly colored gelatin coating solutions are utilized to produce a multi-colored gelatin-coated caplet.

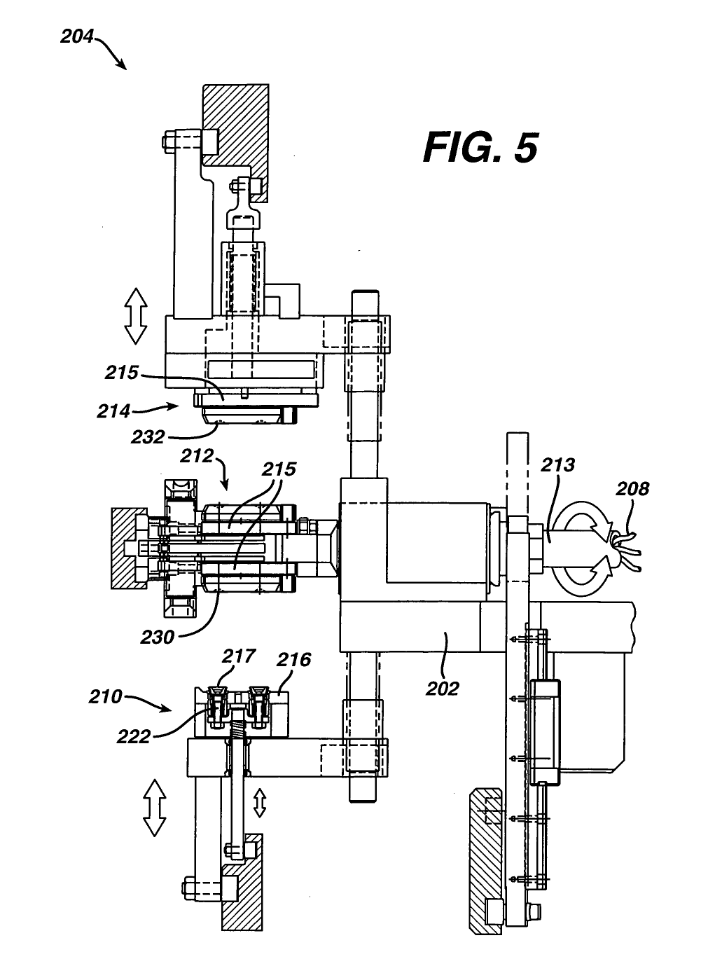

The reservoirs 206 may be mounted to the rotor 202 such that they rotate with the rotor 202, or be stationary and connected to the rotor via a rotary union 207 as shown in FIG. 3. The reservoirs 206 can be heated to assist the flowable material in flowing. The temperature to which the flowable material should be heated of course depends on the nature of the flowable material. Any suitable heating means may be used, such as an electric (induction or resistance) heater or fluid heat transfer media. Any suitable tubing 208 may be used to connect the reservoirs 206 to the mold unit 204. In an embodiment, tubing 208 extends through each of the shafts 213 as shown in FIG. 5 to each of the center mold assemblies 212. Tubing 208 can also be heated to ensure optimal fluid flow. Tubing 208 terminates at material feed plates 215, which serve to distribute flowable material to each mold assembly 212 and 214.

One embodiment of a mold unit 204 is shown in FIG. 5. The mold unit 204 includes a lower retainer 210, an upper mold assembly 214, and a center mold assembly 212. Each lower retainer 210, center mold assembly 212, and upper mold assembly 214 are mounted to the rotor 202 by any suitable means, including but not limited to mechanical fasteners. Although FIG. 5 depicts a single mold unit 204 all of the other mold units 204 are similar.

Lower retainer 210 and the upper mold assembly 214 are mounted so that they can move vertically with respect to the center mold assembly 212. The center mold assembly 212 is rotatably mounted to the rotor 202 such that it may rotate 180 degrees.

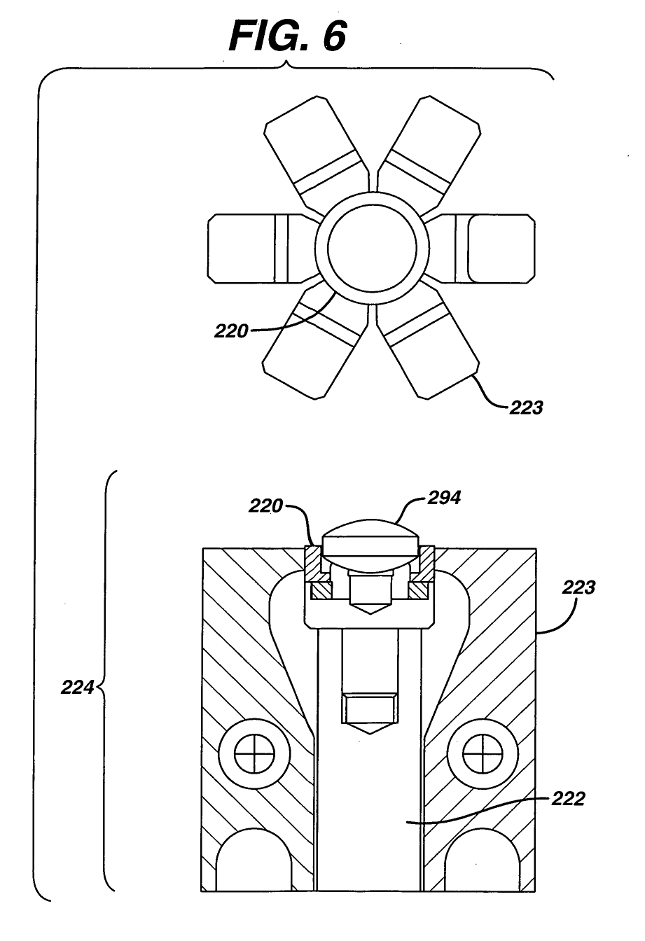

Lower retainer 210 is mounted to the rotor 202 in any suitable fashion and comprises a lower carrier plate 216 and a dosage form holder 217. Each dosage form holder can be connected to the plate by any one of a variety of fastening techniques including without limitation snap rings and groves, nuts and bolts, adhesives and mechanical fasteners. In one embodiment, each dosage form holder 217 includes a gripping device 224 having an elastomeric collet 220, a center support stem 222 and a plurality of flexible fingers 223, all shown in greater detail in FIG. 6.

Center support stem 222 establishes the vertical position of the dosage form. The elastomeric collet 220 masks and seals the periphery of the dosage form. Each elastomeric collet 220 mates with a corresponding portion of center mold assembly 212 in order to create a seal around the dosage form. Although the elastomeric collets can be formed in a variety of shapes and sizes, in one embodiment the elastomeric collets are generally circular and have a corrugated inside surface. The elastomeric collets can optionally further comprise vent holes for air to pass through as flowable material is injected over the top portion of the dosage form. The vent holes are relatively small so that the flowable material injected over the dosage form from the center mold assembly 212 will not flow therethrough.

Moveable fingers 223 are disposed about elastomeric collet 220. Moveable fingers 223 are mounted within the lower retainer 210 by any suitable means and are attached to the center support stem 222 to move up and down with the movement of center support stem 222. Moveable fingers 223 can be coupled to center support stem 222 by any of a variety of fastening techniques. In a preferred embodiment shown, moveable fingers 223 are metal and rotate outward when pushed out, so that a dosage form can be received by or released from an elastomeric collet 220. Moveable fingers 223 move radially inward when retracted by center support stem 222 to hold the dosage form within elastomeric collet 220 firmly. Since the fingers move radially inward they also provide a centering function. Moveable fingers 223 fit tightly in place and a seal is created around core 294 when lower retainer 210 is mated with center mold assembly 212. When an uncoated core 294 is being transferred to lower retainer 210 or a partially coated core 294 is being transferred from lower retainer 210 to center mold assembly 212, center support stem 222 moves to an upward position and moveable fingers 223 expand radially outward. Expansion of the moveable fingers 223 allows elastomeric collet 220 to expand.

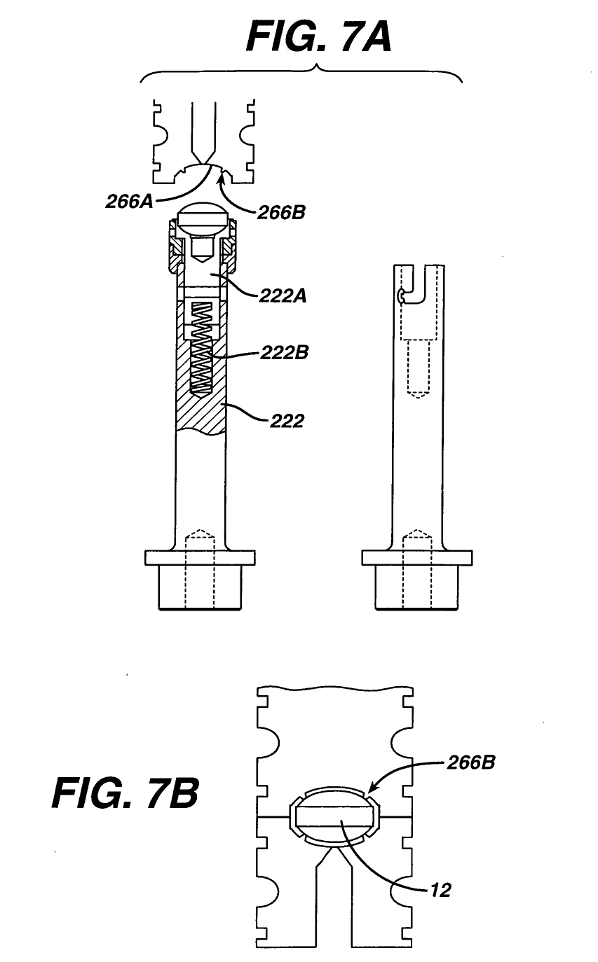

In one embodiment, as exemplified in FIGS. 7A and 7B, center support stem 222, contains a spring means 222b, composed of metal, elastomeric materials, gas bladder, bevel washers, or the like; in communication with a plunger 222A. When the mold closes, at least a portion of the mold surface 266A contacts the core 12, pressing core 12 against the plunger 222A, causing spring 222b to compress. In one such embodiment, the spring applies a pressure to seal core 12 against the mold surface 266A such that when flowable material is injected in the gaps between the core and mold surface, the areas of contact between the core and mold surface [for example the intended openings, logo, or other surface pattern] are thereby masked. In this particular embodiment, mold surface 266A, preferably comprises masking members 266B that are protrusions from the mold surface. The pressure applied by the spring resists an opposite pressure caused by the injection of flowable material that would otherwise tend to separate the core from the masking surface 266A (or masking members 266B thereof). The compliance (or resilience or flexibility) of the spring achieves a relatively uniform masking pressure regardless of variation in core thickness. In particular embodiments in which the mold surface 266A includes projections (masking members 266B) for making openings or indentations in the molded shell or dosage form, the compliance of the spring additionally avoids breakage of the core which may occur due to excessive pressures on the core at the points of contact with masking members 266B or mold surface 266A caused by variation in core thickness.

In another embodiment, a debossed core 12, pressed by spring 222b and plunger 222A against a substantially smooth mold surface 266A, will provide gaps that will be filled with flowable material.

In another embodiment, the spring can be designed to (wire diameter, material, and geometry) to provide a lower force than the resultant opposing force of the pressure caused by the influx of flowable material during the injection event in order to create a partial or incomplete masking effect, such as a dimpled surface texture.

In another embodiment, flowable materials having elastic properties such as those selected from the group consisting of gels, rubbers, silicones, and the like) can provide the resilient feature to avoid breakage of the core and provide masking of the desired patterned area, eliminating the necessity for a spring. This particular embodiment is particularly useful in a 2-step molding process in which the first shell portion comprises the elastic or gel-like material, and the second shell portion includes the desired openings or surface pattern.

In another embodiment, the core composition may provide sufficient ductility to avoid breakage under the pressure of the masking members (266B) of the mold surface 266A.

Masking members 266B can be pins, slots, pads, text, or the like.

In an alternate embodiment, molding of the shell can be accomplished in a single injection, eliminating the need for lower retainer 210, half of the center mold 212. Cores are deposited directly into the center mold 212, they rest upon the masking members/protrusions 266B. When upper mold 214 with its mold surface 266A and optional masking members 266B closes, the core will be suspended by any protrusions or masking members or any features on the core or mold surface which create a flow path for the flowable material.

In a preferred embodiment, flowable material is injected from above core 294 and elastomeric collet 220 stops the flow of the flowable material. Consequently, only the portion of core 294 that is above elastomeric collet 220 will be coated when lower retainer 210 and center mold assembly 210 are mated. This permits a first flowable material to be used to coat one part of the dosage form, and a second flowable material to coat the remainder of the dosage form—that portion which is beneath the elastomeric collet. Although elastomeric collet 220 is shaped so that about half of the dosage form will be coated at one time, elastomeric collet 220 can be of any desired shape to achieve a coating on only a certain portion of the dosage form.

When two halves of core 294 are coated with different flowable materials, the two flowable materials may be made to overlap, or if desired, not to overlap. With the present invention, very precise control of the interface between the two flowable materials on the dosage form is possible. Accordingly, the two flowable materials may be made flush with each other with substantially no overlap. Alternatively, the two flowable materials may be made with a variety of edges, for example to allow the edges of the flowable materials to interlock. In one embodiment, the flowable material coats about half of the uncoated compressed dosage form.

Closing the valve 297 prematurely while injecting the first shell portion can create a unique visual feature. This causes the first shell material to cover a portion of the first face of the core. Consequently, when the second shell flowable material is injected, it flows until it is stopped by the edge of the first shell material. The resulting dosage form has the first shell material covering a portion of a first face, and the second shell material covering the second face and the entire bellyband, and a portion of the first face.

Another unique visual or functional feature can be created by placing a gasketing or masking device between the center mold 212 and the upper mold 214 after injection of the first shell portion and prior to closing of the upper mold against the center mold. The bellyband of the resulting dosage form may be uncoated, exposing the core surface. The exposed core surface may have the form of a continuous band, or a pattern, e.g. dots, dashes, variable thickness lines, or shapes.

Referring generally to FIG. 5, simultaneously with the mating of lower retainer 210 and center mold assembly 212, the center mold assembly 212 and upper mold assembly 214 mate to create seals around a previously made and partially coated core 294. Flowable material is injected through the upper mold assembly 214 into mold cavity 288 created by center mold assembly 212 and upper mold assembly 214 to coat the remaining portion of a partially coated core 294 produced in an earlier cycle. It is possible for the parts to meet other than simultaneously. Accordingly, when an uncoated core 294 is being partially coated between the lower retainer 210 and the center mold assembly 212, the remainder of a previously made and partially coated core 294 is being coated between the center mold assembly 212 and upper mold assembly 214. Following this, the lower retainer and the mold assemblies separate. The fully coated core 294 is retained in the upper mold assembly 214, while the partially coated core 294 is retained in the center mold assembly 214. The fully coated core 294 is then ejected from the upper mold assembly 214 and an uncoated core 294 is transferred to the lower retainer 210 while the center mold assembly 212 rotates with the partially coated core 294 as the process then repeats itself.

In one embodiment, the center mold assembly 212 is rotatably mounted to the rotor 202 on an axis that is radial to the rotor. That is, the axis of rotation of the center mold assembly is perpendicular to the axis of rotation of the rotor. The arrangement allows the center mold assembly to rotate 180 degrees (end for end) at a prescribed time while the zero cycle molding module 200 is simultaneously revolving about its vertical axis. Preferably, the center mold assembly 212 is mounted so that it is capable of rotating 180 degrees in either direction.

The center mold assembly comprises a series of back-to-back, identical center mold inserts 230, one of which is shown in FIG. 7. The center mold assembly 212 rotates partially coated dosage forms from their downwardly oriented positions to upwardly oriented positions. The upwardly pointing portions of the dosage forms, which have been coated with flowable material, can now receive the remainder of their coatings once the center mold assembly 212 mates with the upper mold assembly 214. Also, the insert assemblies previously pointing upward now point downward. Thus they are now in a position to mate with the lower retainer 210 to receive uncoated cores. Flowable material can be injected from the reservoir 206 through the tubing 208 to the center mold insert 230.

In an embodiment, compressed air is used to assist in ejection of the coated dosage form from the center mold assembly 212 to the upper mold assembly 214. Although air is preferred for this purpose, the invention is not limited thereto. An alternative ejector means, such as an ejector pin, may be used. The air can be pressurized to a relatively small pressure and can be provided from air banks or the like.

Although center mold assembly 212 is constructed with identical center mold inserts 230 on both sides of its rotary axis, each center mold insert 230 performs a different function depending on whether it is oriented in the up or in the down position. When facing down, the center mold inserts 230 are actuated to inject flowable material to coat a first portion of a dosage form. Center mold inserts 230 that are facing up are presenting partially coated dosage forms to the upper mold assembly 214. During this time, the upward facing center mold inserts 230 are in a neutral position. Prior to the molds opening however, the upward facing center mold inserts 230 are actuated to allow compressed air to enter the center cavity 288. This ejects the now completely coated dosage forms from the upward facing center mold inserts 230. Thus the completed dosage forms remain seated or held in the upper mold assembly 232.

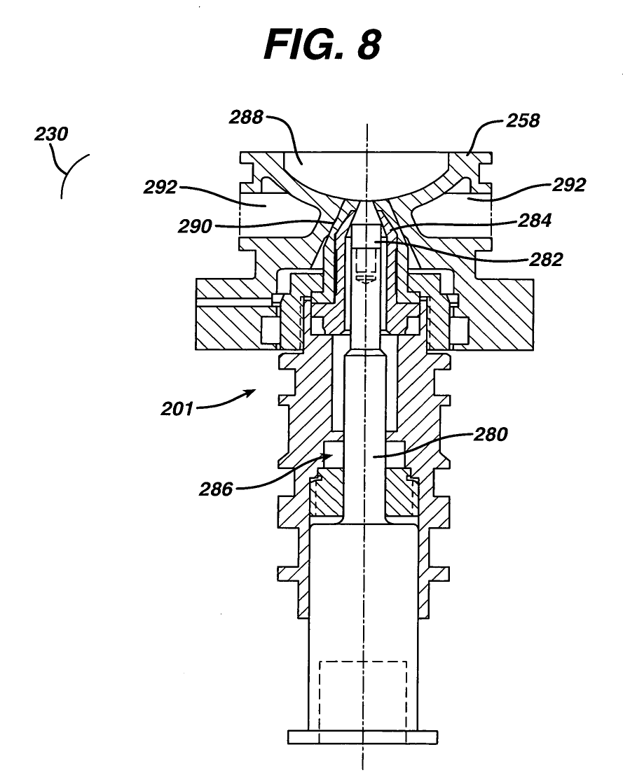

Upper mold assembly 214 is similar in construction to half of center mold assembly 212, and is shown in FIG. 8. Like center mold assembly 212, upper mold assembly 214 directs flowable material to at least partially coat a core. In particular, upper mold assembly 214 has a plurality of upper mold inserts 232 (eight in the preferred embodiment) that mate with corresponding center mold inserts 230. Although upper mold assembly 214 is similar to center mold assembly 212, upper mold assembly 214 does not rotate. Rather, upper mold assembly 214 moves vertically up and down to mate with center mold assembly 212. A seal around each dosage form is preferably created by contact between upward facing center mold insert 230 and an upper mold insert 232. Upper mold cavity 288 is appropriately sized so that the flowable material can flow over the dosage form and provide a coating of the desired thickness. One difference between the upper mold insert 232 and center mold insert 230 is that the upper valve stem 280 and upper conductive sleeve 290 forms part of the ejection mechanism and moves outward rather than inward to eject a dosage form after it has been fully coated. As with center mold assembly 212, compressed air in upper mold assembly 214 can be used to ensure that the coated dosage form does not stick to the upper mold insert 232 when it is ejected. After the coated core is ejected, it may be sent to a transfer device, dryer, or other mechanism. As noted above, the lower retainer and upper mold assembly can be switched without affecting the fundamental operation. Similarly, the order for coating can be modified.

In one embodiment, each mold unit can coat eight compressed dosage forms. Of course, the mold units can be constructed to coat any number of compressed dosage forms. Additionally, the compressed dosage forms are coated with two different colored flowable materials. Any colors can be used. Alternatively, only a portion of the compressed dosage form may be coated while the remainder is uncoated.

Center mold assembly 212 has two molding plates 258, each containing a plurality of center mold inserts 230. Molding plates 258 are affixed to a common carrier plate and rotatably affixed to the zero cycle molding apparatus. Similar upper mold inserts 232 are provided in upper mold assembly 214. Upper mold assembly 214 has a single mold plate containing a plurality of upper mold inserts 232. In an alternative embodiment, the zero cycle molding inserts are provided as a center mold assembly and a lower mold assembly, while the upper unit is a retention unit.

Each zero cycle mold insert has a nozzle system 201 comprising a nozzle tip 284 and a valve body 286 having, as distinct and preferably separable elements, a valve stem 280 and a valve stem tip 282. Nozzle system 201 can optionally include a conductive sleeve 290 that fits over nozzle tip 284 and seats substantially seamlessly into the confines of molding plate 258. Valve stem tip 282 is preferably constructed from an insulative material. Nozzle tip 284 is preferably constructed from an insulative material. Suitable insulative materials include those useful for constructing the valve stem tip 282. Insulative nozzle tip 284 advantageously provides further isolation of the hot gelatin from the cold molding plate 258. Valve stem 280 is preferably constructed from a conductive material. Conductive valve stem 280 advantageously maintains flowable material in a flowable state and allows the flow path to be long enough to accommodate space for material feed plates 215.

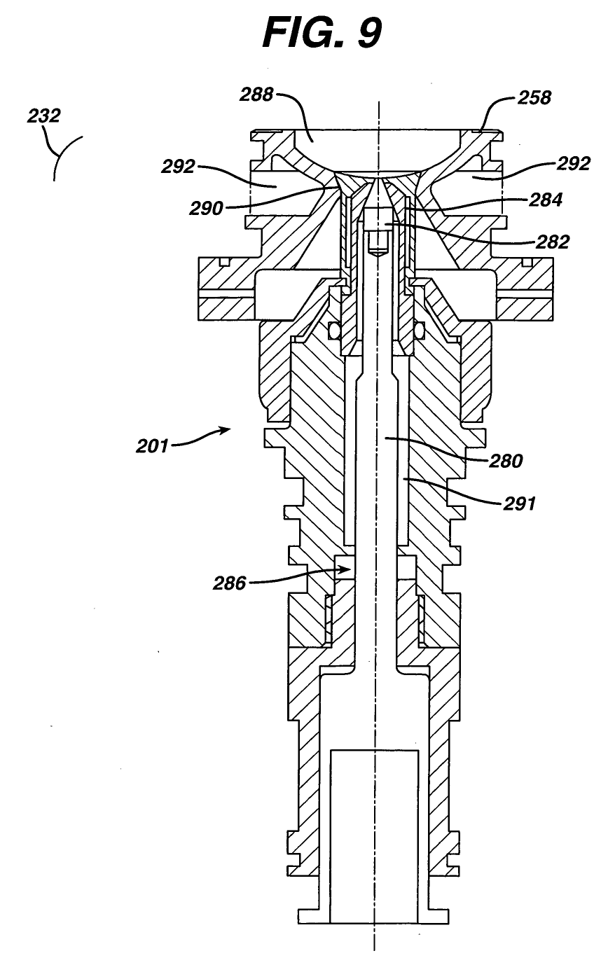

Referring to FIG. 9, conductive sleeve 290 is shown having a tapering conical shape with a truncated and depressed tip and a flanged base 295. The shape of conductive sleeve 290 is not critical other than the need to conform to the desired interior surface of mold cavity 288 and to cover nozzle tip 284. The shape of conductive sleeve 290 for upper mold insert 232 differs since the piece must perform the additional function of acting as an ejector for the finished dosage form upon completion of the zero cycle molding process. Conductive sleeve 290 is provided with an opening 293 through its depressed tip, which forms a part of the interior surface of mold cavity 288. Opening 293 is part of a passageway for the flowable material that results when valve body 286 is in its open position and, when valve body 286 is in its closed position, accepts valve stem tip 282. The end of valve stem tip 282, when valve body 286 is in its closed position against nozzle tip 284, substantially conforms to the shape of the interior surface of mold cavity 288.

Conductive sleeve flow path, 291, shown in FIG. 8, has a path length that is short enough to allow flow of material through the constricted path diameter without allowing for solidification the material exposed to the conductive sleeve. In one embodiment flow path 291 has a length from about 0.015 to about 0.020 inches.

In a preferred embodiment, the end of valve stem tip 282, has an elongated tapered shape. For example, the ratio of the length to diameter at the widest point is at least about 1, or at least about 1.25, say about 1.8. In one embodiment the length to diameter ratio of the tapered portion of the end of valve stem tip is at least about 1. Preferably the included angle between the two sides of the taper is at least about 22 degrees in order to avoid seizing of the tip during valve operation. In one embodiment, the included angle is at least about 45 degrees, say about 60 degrees.

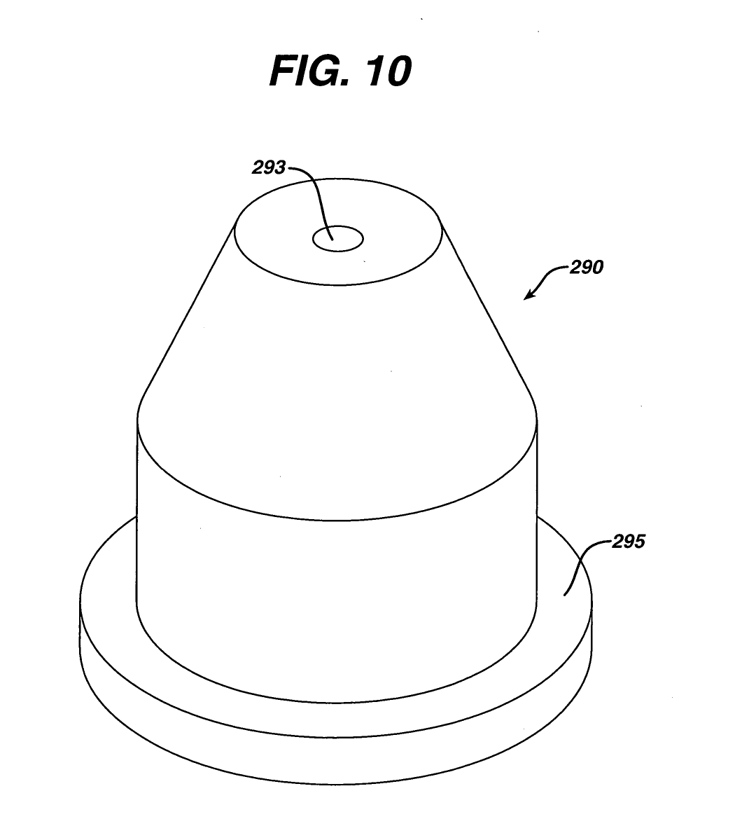

FIG. 10 illustrates both upper mold insert 232 and center mold insert 230. Flowable material is delivered to each nozzle system 201 through tubing 208 into inlet passage 250. The flowable material is transferred via tubing 208 from one or more reservoirs 206 that are maintained at a temperature higher than the softening point or gel point for the flowable material. In another embodiment, tubing 208 is surrounded by a heating system that maintains the flowable material at an elevated temperature. One means for heating tubing 208 includes electric heating. Alternatively, a second tubing layer that carries a heated fluid, such as water, steam or air, can surround tubing 208. The design and configuration of such a heating system is well known in the art. After passing through inlet passage 250, the flowable material enters an interior cavity 252 that surrounds the circumference of valve stem 280 and is sealed by carrier plates 254, nozzle tip 284 and valve stem tip 282 (when closed). Gelatin, shown in black, envelops the exposed portion of core 294 within mold cavity 288 of center mold insert 230. Gelatin is shown flowing into mold cavity 288 of upper mold insert 232 towards completing the coating of core 294.

In order for flowable material to be transferred from interior cavity 252 into mold cavity 288, valve body 286 moves such that valve stem tip 282 is no longer abutting against nozzle tip 284. As valve stem tip 282 separates from nozzle tip 284, a gap 256 develops along a section of valve stem 280 and valve stem tip 282 through which the flowable material passes into mold cavity 288. It should be noted that a portion of gap 256 exists between valve stem 280 and nozzle tip 284 even when nozzle system 201 is in the closed position. This gap gradually tapers to a seal in the closed position.

Molding plate 258 provides the foundation and contours for interior surfaces for mold cavity 288. Molding plate 258 also transfers heat from the flowable material in mold cavity 288 to a heat transfer fluid provided in channels 292 of molding plates 258 for mold assemblies 212 and 214. It will be appreciated that molding plate 258 will not have a uniform temperature. Rather, a temperature gradient will exist along its cross-section from the surfaces of channels 292 for the heat transfer fluid to mold cavity 288. The gradient may be small or large depending on the geometry, time of operation and heat transfer characteristics. Molding plates 258 are constructed from materials having at least good thermal conductivity to enable efficient heat transfer from the flowable material introduced into mold cavity 288 to an associated cooling system described below. Stainless steel has been found to be satisfactory for this purpose. The thermal conductivity, however, should not be too high as to cause the flowable material to harden before fully coating the desired portion of the core.

In contrast, the inventors discovered that the valve stem 280, which is the valve body 286 excluding the valve stem tip 282, should be constructed from materials having at least high, more preferably very high, thermal conductivity. It will be appreciated that tubing 208 can be heated to maintain optimal flow characteristics. However, once flowable material enters interior cavity 252, significant hurdles exist to introducing further heat energy. Hence, without being bound by theory, it is thought that valve stem 280 having at least high thermal conductivity allows for the transfer of heat energy upwards into interior cavity 252 towards mold cavity 288 and ensures good flow characteristics into mold cavity 252.

Suitable materials having at least good thermal conductivity have thermal conductivity at room temperature (23° C.) equal to or greater than 75 BTU-in/ft²-hr-° F. (good), alternatively at least about 500 BTU-in/ft²-hr-° F. (high), and further alternatively at least about 1200 BTU-in/ft²-hr-° F. (very high). Examples of suitable materials having at least good thermal conductivity are aluminum, beryllium-copper, copper, brass, gold and various alloys thereof.

One embodiment uses a copper alloy as the material having sufficiently high thermal conductivity, particularly AMPCO 940. Ampco 940 is an alloy developed by Ampco Metal, Inc. that is approximately 96.4% copper, 02.5% nickel, 00.7% Si, and 00.4% Cr. The following chart lists the thermal conductivities of Ampco 940 along with similar thermally conductive materials.

Thermal Conductivity (BTU-in/ft²-hr-° F.)

| | |
|---|---|
| AMPCO 940 | 1500 |
| Beryllium Copper (5%) | 1500 |
| Aluminum | 960 |
| NO94 | 360 |
| AISI 6150 | 324 |
| H-13 | 204 |
| Stainless Steel | 84 |

The Ampco 940 alloy provides ease of machinability, good adhesion between the substrate material and an electroless Ni layer, an optical finish, sufficient mechanical strength to withstand the pressures (8000-14,000 psi) applied during the injection molding process without deformation, and high thermal conductivity.

While mold plates 258 and valve stem 280 are constructed from materials having at least good thermal conductivity, nozzle tip 284 and valve stem tip 282 are constructed from or coated with materials having no more than low thermal conductivity. The parts are made in manners known per se. Examples of suitable non-conductive or thermally insulative materials are polymeric materials, such as polyetherimides, polyimides, polyether amides, poly-ether-ether ketones, acetals, polyamide-imides, polybenzoimidazoles; ceramics, such as tungsten, modified tungsten, such as thoriated tungsten, thoria, Aerogel. In an alternative embodiment, the respective parts are coated with Teflon (polytetrafluoroethylene), ceramics, and polymeric materials noted above.

Suitable thermally non-conductive or insulative materials have a thermal conductivity at 23° C. that does not exceed 4 BTU-in/ft²-hr-° F. (low), alternatively not greater than 2 BTU-in/ft²-hr-° F., further alternatively less than about 1 BTU-in/ft²-hr-° F. (very low). One embodiment uses Ultem 1000 (unfilled), which is a polyetherimide polymeric material commercially available from General Electric and associated distributors. The following chart lists the thermal conductivities of Ultem along with similar thermally insulative materials.

Thermal Conductivity (BTU-in/ft²-hr-° F.)

| | |
|---|---|
| Ultem 1000 (unfilled) | 0.85 |
| Cycolac GSM | 1.22 |
| Delrin | 1.60 |
| Lexan | 1.35-1.53 |

The mold assemblies, particularly mold plates 258, are maintained at a temperature below the melting or gel temperature of the flowable material. A heat sink and temperature control system are provided to regulate the temperature of the mold assemblies. Examples of heat sinks include but are not limited to chilled air, Ranque Effect cooling, and Peltier effect devices. Electrically powered Freon chillers provide the heat sink for the heat transfer fluid.

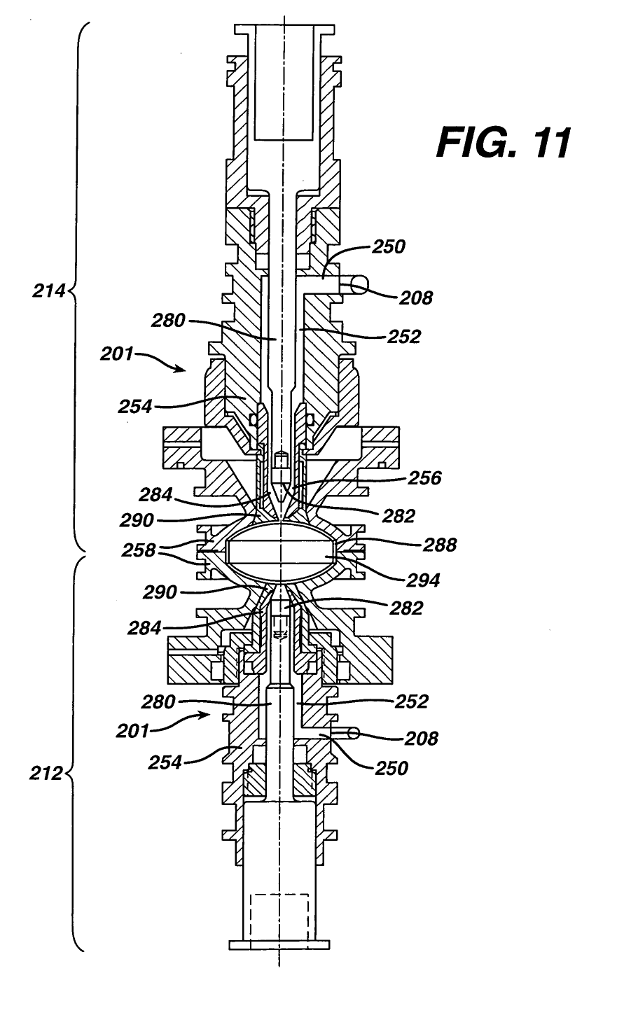

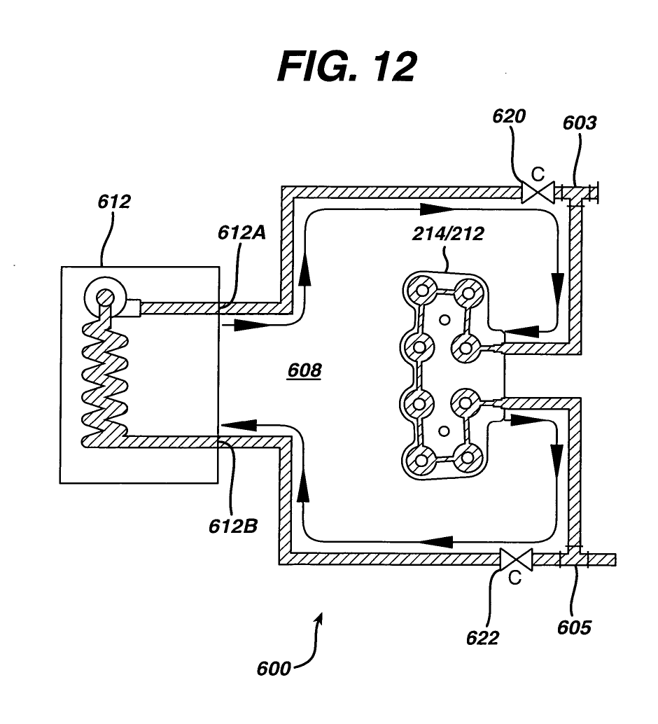

FIG. 11 depicts a temperature control system 600 for the center mold assemblies and upper mold assemblies. Although only one mold assembly 214/212 is depicted, all mold assemblies are connected to the temperature control system in a similar fashion. Tubing system 606 includes a cold loop 608 for cooling mold assembly 214/212. Defined within the flow passageway between fitting 603 and fitting 605 is a flow path in the mold assembly 214/212. An alternative flow pattern that has been found to produce enhanced temperature control employs a single inlet passageway that splits into two distinct pathways, each pathway flowing separately in the vicinity of four mold cavities and exiting separately from the mold assembly. Valves 620 and 622, which may be solenoid or mechanically operated, control the flow of cool heat transfer fluid through the mold assembly 214/212. The system also includes a chiller 612, which provides a chilled fluid source for the cold loop. Outlet ports 612A and inlet ports 612B of the chiller can be connected to multiple molds, so that a single chiller can support all of the upper molds 214 and center molds 212.

Valves 620 and 622, when the molds are in operation, start the flow of chilled heat transfer fluid therethrough. As described above, valves 620 and 622 of the temperature control system can be of various designs known in art, such as spool, plug, ball, or pinch valves. These valves can be actuated by suitable means such as air, electrical solenoids, or by mechanical means such as cam tracks and cam followers. In one embodiment, the valves are pinch valves and are actuated by mechanical cam tracks and cam followers as the zero cycle molding module rotates. Known pinch valves are relatively simple devices comprising a flexible section of tubing and a mechanism that produces a pinching or squeezing action on the tubing. This tubing is compressed or “pinched” to block fluid flow therethrough. Release of the tubing allows fluid to flow. Accordingly, the pinch valve functions as a two-way valve.

Transfer Device

1. Structure of the Transfer Device

Known tablet presses use a simple stationary “take-off” bar to remove and eject tablets from the machine. Since the turrets of these machines rotate at fairly high speeds (up to 120 rpm), the impact forces on the tablets as they hit the stationary take-off bar are very significant. Dosage forms produced on these machines must therefore be formulated to possess very high mechanical strength and have very low friability just to survive the manufacturing process.

The transfer device can be a rotating device, as shown in FIG. 2 and is described more fully in copending application Ser. No. 09/966,939, which is incorporated herein by reference. It comprises a plurality of transfer units 304. It is used for transferring dosage forms or inserts within a continuous process of the invention comprising one or more operating modules, i.e., from one operating module to another. In one embodiment, the coated dosage forms are forcefully ejected from mold cavity for upper mold assembly into a receptacle provided in the transfer device.

The transfer device can take any of a variety of suitable shapes. However, when used to transfer dosage forms or inserts between operating modules of the present invention, transfer device is generally, non-circular, such as a dog bone shaped so that it can accurately conform to the pitch radii of two circular modules, enabling a precision transfer.

Hardening Apparatus

Dosage forms that have been coated with flowable material in the zero cycle molding module are relatively hard compared with dosage forms that have coated using conventional dipping processes. Thus, the amount of drying needed after molding a coating onto a dosage form using the zero cycle molding module is substantially less than that required with known dipping processes. Nevertheless, they may still require hardening, depending upon the nature of the flowable material.

Dosage forms coated in the zero cycle molding module are relatively hard so that they can be tumble hardened relatively quickly. Alternatively, an air dryer may be used. Any suitable dryers may be used. A variety of devices are generally understood in the art.

Specific embodiments of the present invention are illustrated by way of the following examples. This invention is not confined to the specific limitations set forth in these examples, but rather to the scope of the appended claims. Unless otherwise stated, the percentages and ratios given below are by weight.

In the examples, measurements were made as follows.

Coating thickness is measured using an environmental scanning electron microscope; model XL 30 ESEM LaB6, Philips Electronic Instruments Company, Mahwah, Wis. Six tablets from each sample are measured at 6 different locations on each tablet shown in FIG. 1B and described in copending application Ser. No. 09/966,939.

Location 1: center of first major face, t_{c1}

Locations 2 and 3: edges (near punch land) of intersection between first major face and side, t_{c2} and t_{c3}

Location 4: center of second major face, t_{c4}

Locations 5 and 6: edges (near punch land) of intersection between second major face and side, t_{c5} and t_{c6}

Overall dosage form thickness and diameter are measured for 20 dosage forms using a calibrated electronic digital caliper. For diameter, the caliper is positioned at the midsections of the widest point of the dosage form sides.

### EXAMPLE 1

A series of tablets having a molded gelatin coating thereon are made according to the invention as follows.

Part A: Compressed Tablets

The following ingredients are mixed well for 1 minute in a plastic bottle: 2982.6 g [515.5 mg/tablet] of acetaminophen granulation (Compap Grade 3930-PVP3 from Mallinckrodt Inc: 97% acetaminophen), and 17.36 g [3 mg/tablet] of magnesium stearate NF. The resulting dry blend is compressed into tablets on a rotary tablet press [Betapress, Manesty Ltd.] using 0.4375″×0.084″ deep tablet tooling. The resulting tablets have an average weight of 546 mg, thickness of 0.293 inches, and hardness of 18.0 kp. The tablets from Part A are placed by hand into a molding module according to the invention. The tablets are coated with red gelatin-based coating solution on one half thereof, and yellow gelatin-based coating solution on the other half thereof.

The red gelatin-based coating solution is made as follows. Purified water (469.5 g) is heated to 60° C. 275 Bloom Pork Skin Gelatin (262.5 g) is added with mixing. Mixing is continued for 60 minutes at 60° C. Polyethylene oxide WSR N10 (7.5 g) is added with continued mixing for 15 minutes while maintaining the temperature at 60° C. Opatint Red DD-1761 (6.8 g) is added with continued mixing until a uniform color is observed. Then 3.8 gm of phosphoric acid is added, while continually mixing. Mixing is continued until the solution appears uniform. The gelatin solution is held at 60° C. for approximately 1 hour (holding times at this temperature can generally range between about 1 and about 72 hours). The solution is then mixed until uniform (about 5 to 15 minutes), and transferred to a jacketed feed tank equipped with a propeller-type electric mixer. The gelatin-based solution is maintained at 60° C. with continuous mixing at approximately 50 rpm during its use in the molding module.

The yellow gelatin-based coating solution is made as follows. Purified water (458.3 g) is heated to 60° C., and 275 Bloom Pork Skin Gelatin (285 g) is added with mixing. Temperature is maintained at 60° C. with continued mixing for 60 minutes. Opatint Yellow DD-2125 (6.8 g) is added and mixed till a uniform color is observed. The gelatin solution is held at 60° C. for approximately 1 hour (holding times at this temperature can generally range between about 1 and about 72 hours). The solution is then mixed until uniform (about 5 to 15 minutes), and transferred to a jacketed feed tank equipped with a propeller-type electric mixer. The gelatin-based solution is maintained at 60° C. with continuous mixing at approximately 50 rpm during its use in the molding module.

Coating is performed in two steps, the first and second shell portions being applied separately as shown in the flow diagram of FIG. 28B of copending U.S. application Ser. No. 09/966,497. In a first step, first shell portion flowable material [red gelatin-based coating solution], heated to 60° C. to maintain a flowable state in reservoir 206, is injected into the mold cavities created by the closed mold assemblies and a compressed core; the cavity has an overall tablet shell shape of dimensions 0.489″, a draft of 2°×0.050″, a spherical radius of 0.330″, a thickness of 0.020″, and having a mold surface temperature of about 15.3° C. The flowable material is injected at a flow rate of approximately 3 grams per minute using a tank pressure of 80 psi. The first shell portion flowable material then hardens over half the core during a time period of about 1.46 seconds. The mold assemblies separate, the center mold assembly rotates, and then the mold assemblies again close. In a second step, second shell portion flowable material [yellow gelatin-based coating solution], heated to 60° C. to maintain a flowable state in reservoir 206, is injected into the mold cavities at a flow rate of approximately 3 grams per minute using a tank pressure of 85 psi. The mold surface has a temperature of about 15.6° C. The second shell portion flowable material then hardens over half the core during a time period of about 1.4 seconds. The mold assemblies separate, and the finished dosage forms are ejected from the apparatus using an air eject pressure of 80 psi and a mechanical ejection pin.

1. An apparatus for making coated dosage forms comprising:
a) a mold plate;
b) a retention plate;
wherein the mold plate and retention plate define a mold cavity for enclosing a core and having a flow path defined at least in part by an interior surface of said mold plate and the core to be coated; and
c) a nozzle assembly for introducing a flowable material into said mold cavity to coat at least a first portion of said core with said flowable material, said nozzle assembly having a nozzle tip and valve body comprising a valve stem tip, wherein at least a portion of the valve stem tip or nozzle tip are constructed from or coated with a thermally insulative material.

2. An apparatus according to claim 1, wherein the valve body further comprises a valve stem as a distinct element that is constructed from a material having at least high thermal conductivity.

3. An apparatus according to claim 2, wherein the valve stem is constructed from a material having thermal conductivity at 23° C. of at least 1200 BTU-in/ft²-hr-° F.

4. The apparatus according to claim 1, wherein the both the valve stem tip and nozzle tip are constructed from a polymeric material having low thermal conductivity.

5. The apparatus according to claim 3, wherein both the nozzle tip and valve stem tip are constructed from or coated with a material having a thermal conductivity at 23° C. not greater than 2 BTU-in/ft²-hr-° F.

6. The apparatus according to claim 1, wherein the mold plate has an internal surface with protrusions projecting towards the interior of the cavity formed between the mold plate and the retention plate.

7. The apparatus according to claim 6, wherein the core rests upon a spring-biased stem and the protrusions are in contact with the surface of the core.

8. The apparatus according to claim 5 wherein core is in the form of a compressed tablet and the flowable material comprises a gelatin.

9. The apparatus according to claim 1, wherein said flowable material comprises a polymer.

10. The apparatus according to claim 1, wherein said flowable material comprises a material selected from the group consisting of sucrose-fatty acid esters; fats, waxes, fat-containing mixtures, sugars, and low-moisture polymer solutions.

11. An apparatus for making coated dosage forms comprising:
a) a first mold plate;
b) a second mold plate;
wherein the first mold plate and second plate define a mold cavity for enclosing a core and having a flow path defined at least in part by an interior surface of said first mold plate and the core to be coated; and
c) a nozzle assembly in said first mold plate and said second mold for introducing a flowable material into said mold cavity to coat at least a portion of said core with said flowable material, said nozzle assemblies having a nozzle tip and valve assembly comprising a valve stem tip, wherein at least a portion of the valve stem tip or nozzle tip are constructed from or coated with a thermally insulative material.

12. The apparatus according to claim 11, wherein the valve body further comprises a valve stem as a separate element that is constructed from a material having at least high thermal conductivity.

13. The apparatus according to claim 10, wherein both the nozzle tip and valve stem tip are constructed from or coated with a material having a thermal conductivity at 23° C. not greater than 2 BTU-in/ft²-hr-° F., and wherein the valve stem is constructed from a material having thermal conductivity at 23° C. of at least 1200 BTU-in/ft²-hr-° F.

14. The apparatus according to claim 11, wherein said flowable material comprises a polymer.

15. The apparatus according to claim 11, wherein said flowable material comprises a material selected from the group consisting of sucrose-fatty acid esters; fats, waxes, fat-containing mixtures, sugars, and low-moisture polymer solutions.

16. The apparatus according to claim 11, wherein said flowable material is a first flowable material and said mold cavity is a first mold cavity, said apparatus further comprising:
d) a second mold plate for retaining a core and a third mold plate that define a second mold cavity; and
e) a second nozzle assembly having a second nozzle tip and a second valve stem tip for introducing a second flowable material, wherein at least a portion of the second nozzle tip or second valve stem tip are constructed from or coated with a thermally insulative material, wherein said first mold plate and second mold plate are affixed to a common carrier and rotatably mounted onto said apparatus.

17. A method for making a dosage form comprising:
a) providing a core within a mold cavity formed between a mold plate and a retention plate;
b) injecting a flowable material through a nozzle assembly into said mold cavity to coat at least a first portion of said core with said flowable material, said nozzle assembly having a nozzle tip and valve assembly comprising a valve stem tip, wherein at least a portion of the valve stem tip or nozzle tip are constructed from or coated with a thermally insulative material.

18. The method according to claim 17, wherein said flowable material comprises a polymer.

19. The method according to claim 17, wherein said flowable material comprises a material selected from the group consisting of sucrose-fatty acid esters; fats, waxes, fat-containing mixtures, sugars, and low-moisture polymer solutions.

20. The method according to claim 17, wherein said flowable material comprises a gelatin.

21. The method according to claim 17 further comprising:
c) separating said mold plate and retention plate;
d) rotating said mold plate while retaining the partially coated core into alignment with a second mold plate;
e) sealing said mold plate and second mold plate to enclose the partially coated core within a second mold cavity;
f) injecting a flowable material through a second nozzle assembly in said second mold plate into said second mold cavity to coat at least a second portion of said core with said flowable material, said second nozzle assembly having a second nozzle tip and second valve stem tip, wherein at least a portion of the second valve stem tip or second nozzle tip are constructed from or coated with a thermally insulative material.

22. The method according to claim 21 wherein the flowable material comprises a gelatin and at least one mold plate is made from a material having good thermal conductivity and is continually maintained during molding operations at a temperature below the softening point for the selected flowable material, and wherein the valve stem is constructed from a material having thermal conductivity at 23° C. of at least 1200 BTU-in/ft²-hr-° F.

23. A method for making dosage forms comprising:
a) providing within a mold cavity formed between a first mold plate and a second mold plate a core having a coating on a first portion thereof;
b) injecting a flowable material into said mold cavity through a nozzle assembly provided in said first mold plate to coat at least a second portion of said core with said flowable material, said nozzle assembly having a nozzle tip and valve assembly comprising a valve stem tip, wherein at least a portion of the valve stem tip or nozzle tip are constructed from or coated with a thermally insulative material.

24. The method according to claim 23 wherein the flowable material comprises a gelatin and at least one mold plate is made from a material having good thermal conductivity and is continually maintained during molding operations at a temperature below the softening point for the selected flowable material, and wherein the valve stem is constructed from a material having thermal conductivity at 23° C. of at least 1200 BTU-in/ft²-hr-° F.

25. A dosage form produced according to claim 17 wherein the core is in the form of a compressed tablet.

26. The dosage form of claim 25 having a coating of hardened gelatin-containing material with an average thickness not greater than about 400 microns, more preferably about 100 to 300 microns.

27. A dosage form produced according to the method of claim 14 wherein the core is in the form of a compressed tablet.

28. The method according to claim 23, wherein said flowable material comprises a polymer.

29. The method according to claim 23, wherein said flowable material comprises a material selected from the group consisting of sucrose-fatty acid esters; fats, waxes, fat-containing mixtures, sugars, and low-moisture polymer solutions.

30. The method according to claim 23, wherein said flowable material comprises a gelatin.

31. A method of making dosage forms, comprising the steps of:
a) compressing a powder into a compressed core in a compression module;
b) transferring said compressed core to a zero cycle molding module;
c) molding a flowable material around said compressed core in said zero cycle molding module; and
d) hardening said flowable material so as to form a coating over said compressed core;
wherein steps (a) through (d) are linked together such that essentially no interruption occurs between said steps.

32. The method of claim 31, wherein one or more of said steps is performed on a continuous basis.

33. The method of claim 31, wherein said powder contains a pharmaceutical active ingredient.

34. The method of claim 31, wherein said flowable material contains a pharmaceutical active ingredient.

35. The method of claim 31, wherein steps (a) through (d) are performed simultaneously, such that while coatings are being hardened on a first group of cores in step (d), flowable material is being molded around a second group of cores in step (c), a third group of cores are being transferred to said zero cycle molding module in step (b), and a fourth group of cores are being formed in step (a).

36. The method according to claim 31, wherein step (c) comprises the steps of:
(i) molding a first flowable material around a first portion of said compressed dosage form; and
(ii) molding a second flowable material around a second portion of said compressed dosage form.

37. The apparatus according to claim 36, wherein said flowable material comprises a gelatin.

38. The method according to claim 31, wherein said flowable material comprises a polymer.

39. The method according to claim 31, wherein said flowable material comprises a material selected from the group consisting of sucrose-fatty acid esters; fats, waxes, fat-containing mixtures, sugars, and low-moisture polymer solutions.

40. The method according to claim 31, wherein said flowable material comprises a gelatin.
